(19) United States
(12) Patent Application Publication
(10) Pub. No.: US 2006/0129088 A1
(43) Pub. Date: Jun. 15, 2006

(54) Metering pump control system

(76) Inventors: Daniel M. Gill, Macedon, NY (US); Stephen B. Muscarella, West Henrietta, NY (US); Leonid Buchman, Rochester, NY (US); Patrick Miller, Spencerport, NY (US); Cordell E. Claude, Hamlin, NY (US); Dennis C. Heidt, Palmyra, NY (US)

Correspondence Address: HODGSON RUSS LLP, ONE M & T PLAZA, SUITE 2000, BUFFALO, NY, 14203-2391, US

(21) Appl. No.: 11/257,671

(22) Filed: Oct. 25, 2005

Related U.S. Application Data
(60) Provisional application No. 60/621,880, filed on Oct. 25, 2004.

(51) Int. Cl. A61M 31/00

(52) U.S. Cl. 604/67

(57) ABSTRACT

A metering pump system having a human interface device that is selectively mounted to a pump base. In an embodiment of the invention, sensors are provided that monitor characteristics corresponding to the material being pumped, and provide information about those characteristics to a computer which alters pumping parameters based on the characteristics.

### CROSS-REFERENCE TO RELATED APPLICATION

This application claims the benefit of priority to U.S. provisional patent application Ser. No. 60/621,880, filed on Oct. 25, 2004.

### FIELD OF THE INVENTION

The present invention relates to a metering pump, such as those used to dispense chemicals into industrial processes.

### DESCRIPTION OF THE INVENTION

The invention will now be described by way of a non-limiting example, with reference to the attached drawings and diagrams in which:

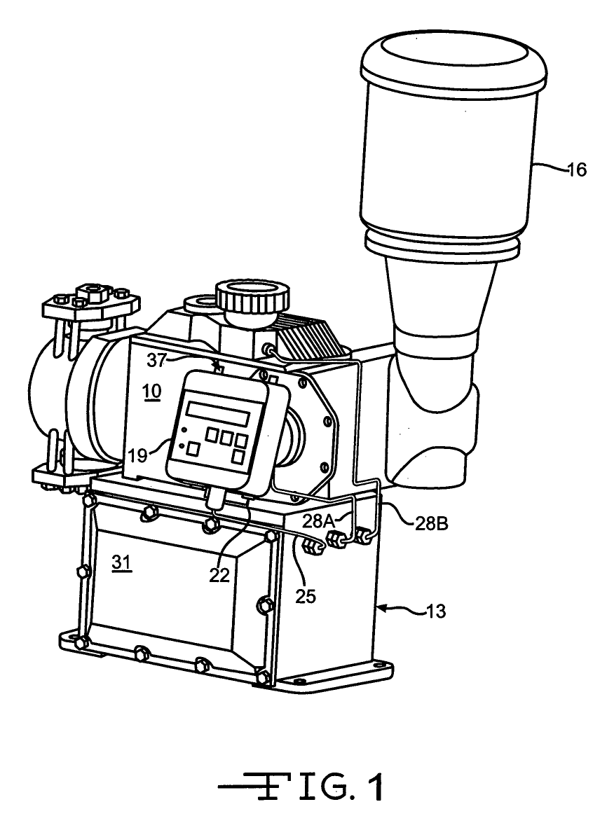

FIG. 1 is a perspective view of a metering pump system according to the invention;

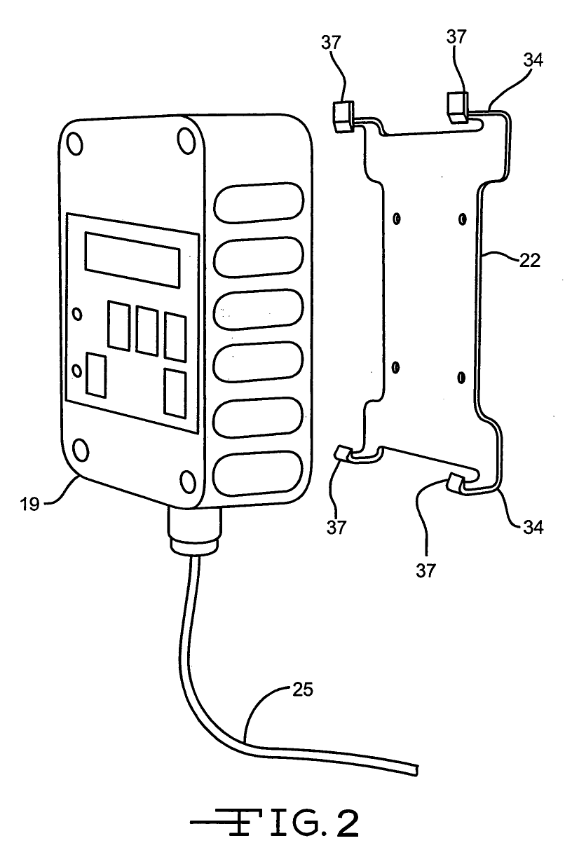

FIG. 2 is a perspective view of a human interface device and bracket according to the invention; and

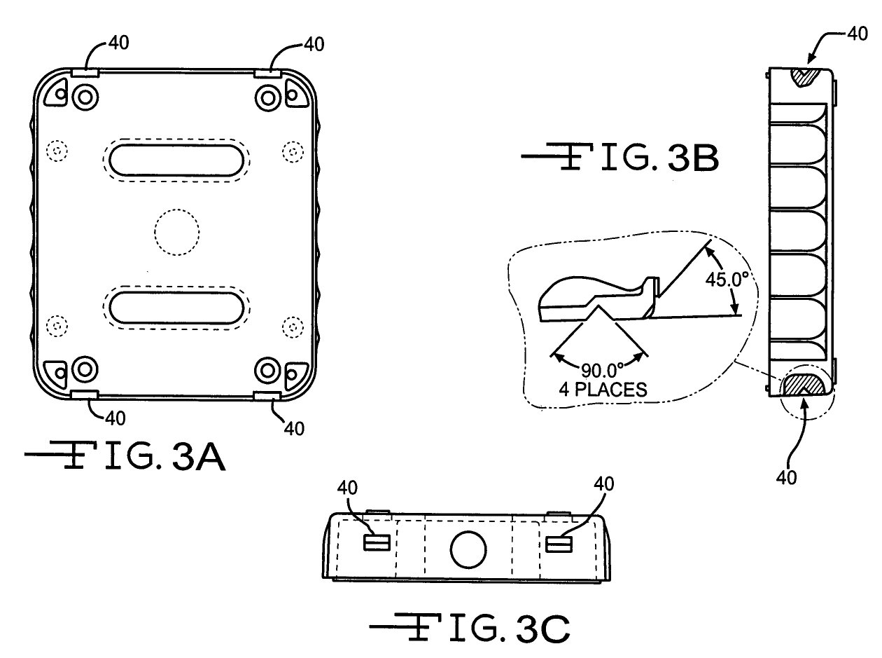

FIGS. 3A, 3B and 3C are front, side and bottom views respectively of a human interface device according to an embodiment of the invention.

There is shown in FIG. 1 an example of a metering pump system according to the invention. The system may include a metering pump 10 mounted on a base 13. The pump 10 may be driven by a motor 16. The pump 10 may be controlled via a human interface device 19 that is selectively attachable/detachable to a bracket 22. FIG. 2 depicts an interface device 19 and bracket 22 that is in keeping with the invention. The bracket 22 may be attached to the pump base 13. The human interface device 19 may be used to communicate changes in the stroke length and/or the motor speed of the metering pump 10, and thereby adjust the amount of material pumped by the metering pump 10, or the timing at which the pump 10 pumps material, or both.

The pump base 13 may serve as a housing for control electronics. The control electronics may accept signals provided by the human interface device 19, interpret those signals from the human interface device 19, and provide control signals to the metering pump 10, motor 16, or both in order to alter the amount of material pumped, timing at which the pump 10 pumps material, or both. The control electronics may be mounted on the inside surface of the maintenance door 31.

Communication between the human interface device 19 and the electronics in the pump base 13, may be via wired or wireless connections. In FIG. 1, there is shown a wired connection in which an input communication line 25 and two output communication lines 28A, 28B are shown. In the embodiment shown in FIG. 1, the input communication line 25 is shown to be quite short, but it should be recognized that the input communication line 25 may be made very long. In the instance where the input communication line 25 is long, a pair of prongs may be provided to allow the input communication line 25 to wound around the prongs, and thereby safely stored.

In the embodiment shown in FIG. 1, output communication line 28A provides control signals to alter the speed at which the pump 10 operates. Output communication line 28B provides control signals to alter the stroke length of the pump 10. The invention is not limited to this arrangement.

The bracket 22 may include flexible arms 34, which terminate in fingers 37. The fingers 37 may engage mating receptacles 40 on the human interface device 19. FIGS. 3A, 3B and 3C show the receptacles. When the fingers 37 are engaged with the receptacles 40, the human interface device 19 is held to the bracket 22. By pulling on the human interface device 19, the arms 34 are caused to flex, thereby moving the fingers 37 out of the receptacles 40, and thereby allowing the human interface device 19 to be removed from the bracket 22. When desired, the human interface device 19 may be engaged with the bracket 22 by pushing the human interface device 19 against the fingers 37, thereby causing the arms 34 to flex enough to allow each finger 37 to seat in a corresponding receptacle 40.

The embodiment of the invention described above allows the pump 10 to be controlled via the human interface device 19, regardless of whether the human interface device 19 is attached to the bracket 22. In this fashion, the human interface device 19 may be stored on the bracket 22, but allow an operator to remove the human interface device 19 from the bracket 22 and control the pump 10 from a location that is distant from the pump 10. This may improve safety for the operator, for example where the pump 10 is used to pump hazardous material, or where the pump 10 is in a noisy environment.

It will be recognized that variations may be made to the embodiment of the invention described above, and yet be within the scope of the invention. For example, the human interface device 19 may be used to communicate information to an operator. That information may include data that corresponds to whether the pump 10 is pumping the intended material, the concentration of the material being pumped, or both. For example, a sensor may be installed in the pumping system that communicates to the control electronics whether a particular chemical is being pumped or whether air is being pumped. This may be particularly useful when it is difficult to check whether the supply of chemical has run out. The control electronics may then provide this information to an operator via the human interface device 19. Also, the control electronics may include a microprocessor programmed to alter the pumping parameters (e.g. motor speed and/or stroke length) based on the information provided by the sensors.

U.S. provisional patent application Ser. No. 60/621,880 discloses additional details about the invention and additional embodiments of the invention. The disclosure of that patent application is incorporated by this reference.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A metering pump system having a human interface device that is selectively mounted to a pump base.

2. The system of claim 1, wherein the human interface device communicates with control electronics via wired or wireless means.

3. The system of claim 1, wherein the human interface device communicates changes to the stroke length and/or motor speed, or both.

4. A metering pump system having sensors that monitor characteristics corresponding to the material being pumped, and provide information about those characteristics to a computer which alters pumping parameters based on the characteristics.

5. The system of claim 4, wherein the characteristics include whether the intended material is pumped.

6. The system of claim 4, wherein the characteristics include the concentration of the material being pumped.
